(19) United States
(12) Patent Application Publication
(10) Pub. No.: US 2007/0275035 A1
(43) Pub. Date: Nov. 29, 2007

(54) Minimally Invasive Medical Implant Devices for Controlled Drug Delivery

(75) Inventors: Stephen J. Herman, Andover, MA (US); John T. Santini, North Chelmsford, MA (US); Mark A. Staples, Cambridge, MA (US)

Correspondence Address: SUTHERLAND ASBILL & BRENNAN LLP, 999 PEACHTREE STREET, N.E., ATLANTA, GA, 30309, US

(73) Assignee: MICROCHIPS, INC., Bedford, MA (US)

(21) Appl. No.: 11/753,330

(22) Filed: May 24, 2007

Related U.S. Application Data
(60) Provisional application No. 60/803,107, filed on May 24, 2006.

(51) Int. Cl. A61K 9/14; A61M 31/00

(52) U.S. Cl. 424/426; 424/489; 977/906; 604/500

(57) ABSTRACT

Implantable medical devices and methods of use are provided for controlled drug delivery. The devices may include an elongated body dimensioned to facilitate minimally invasive and complete implantation into a patient, wherein the elongated body portion comprises a first, closed end portion, a second closed end portion and an intermediate portion disposed between and connected to the first closed end portion and the second closed end portion; two or more discrete reservoirs disposed in the elongated body portion, each reservoir having one or more openings; a drug formulation, which comprises at least one drug, disposed in the two more discrete reservoirs; and reservoir caps closing off the one or more openings of each reservoir, wherein release of the drug in vivo is controlled at least in part by the in vivo disintegration or permeabilization of the discrete reservoir caps.

### CROSS-REFERENCE TO RELATED APPLICATIONS

This application claims benefit of U.S. Provisional Application No. 60/803,107, filed May 24, 2006. This application is incorporated herein by reference.

### BACKGROUND OF THE INVENTION

This invention is generally in the field of devices and methods for controlled exposure or release of reservoirs contents, particularly medical implant devices.

Implantable medical devices for the delivery of drugs to patients over extended periods of time are known. U.S. Pat. No. 5,797,898, U.S. Pat. No. 6,527,762, and U.S. Pat. No. 6,491,666, and U.S. Pat. No. 6,976,982 describe devices for the storage and controlled release of drug formulations from multi-reservoir devices. One category of the devices provide passive controlled release of multiple, individual doses of drug. The reservoirs contain a release system which comprises a drug. That is, the release system of each reservoir can be individually “programmed,” e.g., formulated, to provide selected kinetics of drug release, controlling both the time at which release is initiated and the rate at which the drug is released. Release from different reservoirs can be staggered to provide complex release profiles. Reservoir caps may be provided to close off reservoir openings until such time as release of the drug is desired. Reservoir caps are designed to passively disintegrate or become permeable in vivo to initiate drug release. It would be desirable to provide improved designs of such implantable medical devices, such as for therapeutic or prophylactic treatments. It would also be desirable to provide a device for precise, local (e.g., pinpoint) delivery of one or more drugs over an extended period precise to selected tissue structures in vivo, particularly a device that can be implanted into a patient by a minimally invasive technique. It would be further desirable to provide passively controlled release of one or more drugs with a range of release profiles, from an implantable medical devices that is sized and shaped for administration to a variety of tissue smaller or difficult-to-reach tissue structures, particularly using a minimally invasive procedure.

### SUMMARY OF THE INVENTION

In one aspect, minimally invasive implantable medical devices are provided for controlled drug deliver. In one embodiment, the device may include an elongated body dimensioned to facilitate minimally invasive and complete implantation into a patient, wherein the elongated body portion comprises a first, closed end portion, a second, closed end portion, and an intermediate portion disposed between and connected to the first closed end portion and the second closed end portion; two or more discrete reservoirs disposed in the elongated body portion, each reservoir having one or more openings; a drug formulation, which comprises at least one drug, disposed in the two more discrete reservoirs; and reservoir caps closing off the one or more openings of each reservoir, wherein release of the drug in vivo is controlled at least in part by the in vivo disintegration or permeabilization of the discrete reservoir caps.

In a preferred embodiment, the intermediate portion is substantially tubular in shape and the first, closed end portion includes a rounded end. The device may be suitable for passing through a trochar, hollow needle, or catheter.

In one embodiment, the elongated body is formed of a polymer. In another embodiment, the elongated body is formed of a metal. The discrete reservoirs disposed in the elongated body portion may be microreservoirs. The reservoir caps closing off the one or more openings may include a polymer which disintegrates in vivo. At least two of the reservoir caps may differ from one another in composition or thickness or both.

The drug formulation disposed in the reservoirs may be in a solid form. In a particular embodiment, the drug formulation includes microparticles or nanoparticles of a drug. Alternatively, the solid drug formulation in each reservoir may be in a monolithic structure form. In one embodiment, the drug formulation includes a protein, a peptide, or a nucleic acid. The drug formulation may be a vaccine. The drug formulation also may be a chemotherapeutic agent or a hormone.

In a preferred embodiment, at least one of the reservoirs may have a first opening and an opposing second opening located distal to one another in the traverse direction in the intermediate portion of the elongated device body. In certain embodiments, at least one of the reservoirs may extend into the elongated body in a substantially traverse direction to a depth which is more than 50% of the maximum traverse width of the intermediate body portion.

In a particular embodiment, the device includes a linear array of three or more reservoirs, each containing a drug formulation and having a discrete reservoir cap closing off at least one opening of each reservoir. In another embodiment, the elongated body defines a plurality of reservoirs about the circumference of the device and wherein the reservoirs extend the substantial length of the elongated body.

In yet another aspect, implantable medical devices are provided that may include: a first solid unit of a drug formulation which comprises at least one drug, the first solid unit have a proximal end, a distal end, and one or more longitudinal exterior surfaces therebetween; a second solid unit of the drug formulation which comprises the at least one drug, the second solid unit have a proximal end, a distal end, and one or more longitudinal exterior surfaces therebetween; a layer of a first membrane material coating the longitudinal exterior surfaces of the first solid unit; a layer of a second membrane material coating the longitudinal exterior surfaces of the second solid unit; and a substantially impermeable seal member sandwiched between the proximal end of the first solid unit and the distal end of the second solid unit, such that the first solid unit, seal member, and second solid unit are connected together aligned in an elongated rod shaped form, which is dimensioned to facilitate minimally invasive and complete implantation into a patient, wherein release of the drug in vivo is controlled at least in part by the in vivo disintegration or permeabilization of the first and second membrane materials.

In one embodiment, the release of the drug from the first solid unit occurs at a different time or rate than the release of the drug from the second solid unit. The first and second units may each have a cylindrically shaped exterior surface, and the seal member may be disk shaped.

In a particular embodiment, the seal member includes a non-degradable polymer. The first membrane material, the second membrane material, or both membrane materials may include a biodegradable polymer. The drug formulation may further include one or more pharmaceutically acceptable excipient materials, along with the drug itself.

In one embodiment, the device includes three or more of the membrane coated solid units of drug formulation and two or more of the seal members. The device may further include a plurality of rods connecting the seal members.

In yet another aspect, methods are provided for the administration of a drug to a patient. In one embodiment, the method includes injecting the implantable medical device into a tissue site in a patient; and releasing the drug from the medical device to the tissue site. In one example, the step of injecting may be subcutaneous or intramuscular. In particular embodiments, the step of injecting includes passing the implantable medical device through the hollow bore of a needle, cannula, catheter, or trocar. Examples of tissue sites include the brain, the peritoneal cavity, an intracranial cavity, or the pleural space. In one case, the tissue site may be a tumor mass.

### BRIEF DESCRIPTION OF THE DRAWINGS

FIG. 1 is an external, perspective view of an assembled, implantable medical device according to one embodiment of the invention.

FIG. 2 is an external, perspective view of the embodiment of the implantable medical device shown in FIG. 1, but with the reservoir caps and drug formulations removed.

FIG. 3 is an exploded, perspective view of one embodiment of an implantable medical device.

FIG. 4 illustrates various embodiments of pre-formed drug formulations sized and shaped to fit inside elongated reservoirs of an implantable medical device according to one embodiment of the invention.

FIGS. 5a-5d are cross-sectional views that illustrate possible embodiments of reservoir cap disintegration.

FIGS. 6a-b, are top-view and cross-sectional views, respectively, of an elongated implant device according to certain embodiments of the invention. FIG. 6b illustrates non-limiting examples of possible reservoir shapes and reservoir cap positions.

FIGS. 7a-b are perspective and cross-sectional views, respectively, of one embodiment in which the reservoir is recessed into the elongated implant device,

FIG. 8 is a perspective and partially exploded view of one embodiment of an elongated implant device which includes two reservoirs.

FIG. 9 illustrates a process for making one embodiment of an elongated implantable medical device.

FIGS. 10a-b illustrate a perspective/transparent view and a cross-sectional view, respectively, of one embodiment of an elongated implant device in which a plurality of impermeable discs are connected with support rods.

FIGS. 11a-b illustrate cross-sectional views of one embodiment of an elongated medical implant which includes three elongated, longitudinally oriented reservoirs.

### DETAILED DESCRIPTION OF THE INVENTION

Medical implantable reservoir devices have been developed that include at least one reservoir for controlled release of a drug, wherein the device facilitates minimally invasive and complete implantation. In an exemplary embodiment, the implantable reservoir device includes an elongated substrate to allow complete insertion through a conventional needle (e.g., large gauge hypodermic needle) or with a trocar into a specific location in the body of a patient, such as a human or other mammal in need of treatment or prophylaxis. The targeted tissue location may be, for example, a lumen other than the vasculature, subcutaneous space, intramuscular, in a specific organ/tumor mass, peritoneal cavity, intracranial cavity or brain tissue, or pleural space. The entire implantable reservoir device is completely implanted into the targeted tissue or tissues, as distinct from a catheter, at least a portion of which generally protrudes/extends from the body.

As used herein, the terms “comprise,” “comprising,” “include,” and “including” are intended to be open, non-limiting terms, unless the contrary is expressly indicated.

Device Body and Reservoirs

The device includes two or more discrete reservoirs located or defined in a device body portion. In an exemplary embodiment, the elongated substrate may be substantially tubular in shape with substantially rounded ends. As used herein, the term “elongated” is used broadly to include without limitation a shape wherein the longitudinal axis is substantially longer than a lateral axis of the device. It should be understood that there are many elongated implantable device geometries that could embody the concept of employing discrete, reservoirs that contain a drug formulation until a specific time for release and the device described herein is not limited to a substantially tubular design.

The total volume of the reservoirs desirably is a large fraction of the volume of the whole implant device, so that as much drug volume as possible is packaged in as small an elongated body as possible. Therefore, a reservoir desirably may extend substantially into or through the body structure. The shape and dimensions of the reservoir, along with the number and size of the reservoir openings in the surface of the device body, can be selected to influence the rate of drug diffusion from a reservoir.

The elongated body portion can be fabricated from a non-degradable (e.g., non-disposable) material and remain in the patient, or it may be retrieved after release of the drug formulation. Alternatively, it could be fabricated from a degradable material that erodes or degrades after the covers (i.e., reservoir caps) have degraded and the reservoir contents have been released. The elongated body portion may be formed of a biocompatible ceramic, silicon, metal (e.g., titanium, stainless steel), polymer, or a combination thereof. The polymer may be a biodegradable or bioerodible polymer or copolymer. Alternatively, the polymer may be non-degradable. Non-limiting examples of materials of construction include poly(lactic acid)s, poly(glycolic acid)s, and poly(lactic-co-glycolic acid)s, and degradable poly(anhydride-co-imides), polytetrafluoroethylenes, polyesters, and silicones, The body portion defining the reservoir may be a composite or multilayer structure.

In an exemplary embodiment, the body portion can be modular. Each module may include one or more reservoirs with one or more reservoir caps. Multiple covered reservoirs may then be connected together, e.g., linked end-to-end for insertion, providing flexibility to the clinician with respect to timing and dosage for the prescription. The linkage between modules may be substantially rigid, or may permit flexing, movement between modules. The connection between modules may be releasable or non-releasable.

The elongated body portion may be substantially rigid. It may desirably have rounded ends and without sharp edges on any exterior surface. In various embodiments, the reservoirs are discrete, non-deformable, and disposed in an array in the body. The reservoir openings may be along one side of the device body, or in a preferred embodiment, may be provided around the longitudinal surfaces to release drug in multiple directions. The elongated body may include tens or hundreds of reservoirs arrayed across exterior surface.

In one embodiment, the reservoirs are microreservoirs. A “microreservoir” is a reservoir suitable for storing and releasing/exposing a microquantity of material, such as a drug formulation. The term “microquantity” refers to volumes from 1 nL up to 500 μL. In one embodiment, the microreservoir has a volume equal to or less than 500 μL (e.g. less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). In one embodiment, the microquantity is between 1 nL and 1 μL. In another embodiment, the microquantity is between 10 nL and 500 nL. In still another embodiment, the microquantity is between about 1 μL and 500 μL. In another embodiment, the reservoirs are macroreservoirs. The “macroreservoir” is a reservoir suitable for storing and releasing/exposing a quantity of material larger than a microquantity. In one embodiment, the macroreservoir has a volume greater than 500 μL (e.g., greater than 600 μL, greater than 750 μL, greater than 900 μL, greater than 1 mL, etc.) and less than 5 mL (e.g., less than 4 mL, less than 3 mL, less than 2 mL, less than 1 mL, etc.). Unless explicitly indicated to be limited to either micro- or macro-scale volumes/quantities, the term “reservoir” is intended to encompass both.

In one embodiment, the device body may include one or more features for securing the device at the site of implantation. For instance, it would be desirable to maintain the device within the tumor or other space where local drug delivery is needed. Examples of such features include suture loops, retention barbs, screws, hooks, etc. These features may be integral with the substrate or attached thereto following implantation. In one case, movable features may be includes which do not extend from the smooth surface of the device body (i.e., substrate) until after implantation. For instance, a water-swellable material can be provided behind a barb feature, which material will swell in vivo to drive the tip end of the barb out in a protruding position.

Drug Formulation, other Reservoir Contents

The two or more reservoirs each include at least one drug formulation contained therein. The drug formulation is a composition that comprises a drug. As used herein, the term “drug” includes any therapeutic or prophylactic agent (e.g., an active pharmaceutical ingredient or API). The drug formulation may include one or more pharmaceutically acceptable excipients, which are known in the art.

The drug formulation may be in essentially any form, such as a pure solid or liquid, a gel or hydrogel, a solution, an emulsion, a slurry, or a suspension. In a preferred embodiment, the drug formulation is in a monolithic, dry solid form, or in the form of a collection of particles (e.g., microparticles or nanoparticles), particularly for purposes of maintaining or extending the stability of the drug over a commercially and medically useful time, e.g., during storage in a drug delivery device until the drug needs to be administered. These solid forms may be provided by lyophilization of a drug solution or suspension directly in the reservoirs. Alternatively, prefabricated pellets that are approximately the size of the individual reservoirs may be formed outside the reservoirs (e.g., in a mold) and then transferred and deposited into the reservoirs, e.g., by a pick-and-place technique. Alternatively, the drug formulation may be in a gel, liquid, or suspension form. The particular formulation in the reservoirs of a single device may be the same as or different from one another across a plurality of the reservoirs.

The drug formulation may include a drug in combination with other materials to control or enhance the rate and/or time of release from an opened reservoir. In various embodiments, the drug formulation further includes one or more matrix materials. In one example, the matrix material comprises one or more synthetic polymers. Exemplary materials include synthetic polymers, such as PLGA, PEG, PLLA, and/ or naturally occurring polymers such as hyaluronic acid, chitosan, and alginate. The natural-occurring polymers may or may not be crosslinked by methods known to the art. In another example, the one or more matrix materials comprise a biodegradable, bioerodible, water-soluble, or water-swellable matrix material. In one embodiment, the therapeutic or prophylactic agent is distributed in the matrix material and the matrix material degrades or dissolves in vivo to controllably release the therapeutic or prophylactic agent. The therapeutic or prophylactic agent may be heterogeneously distributed in the reservoir or may be homogeneously distributed in the reservoir. This matrix material can be a “release system,” as described in U.S. Pat. No. 5,797,898, the degradation, dissolution, or diffusion properties of which can provide a method for controlling the release rate of the drug molecules. In one embodiment, the drug is formulated with an excipient material that is useful for accelerating release, e.g., a water-swellable material that can aid in pushing the drug out of the reservoir and through any tissue capsule over the reservoir. In another embodiment, the drug is formulated with one or more excipients that facilitate transport through tissue capsules. Examples of such excipients include solvents such as DMSO or collagen- or fibrin-degrading enzymes.

The drug may comprise small molecules, large (i.e., macro-) molecules, or a combination thereof. In one embodiment, the large molecule drug is a protein or a peptide.

Representative examples of suitable drugs include vaccines, vectors for gene therapy, polypeptides, nucleic acids (DNA, siRNA), interferons, antibodies, anti-inflammatories, hormones, and chemotherapeutic agents. In various other embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. In one embodiment, the drug is a protein. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). In one embodiment, the drug is a gonadotropin-releasing (LHRH) hormone analog, such as leuprolide. In another exemplary embodiment, the drug comprises parathyroid hormone, such as a human parathyroid hormone or its analogs, e.g., hPTH(1-84) or hPTH(1-34). In a further embodiment, the drug is selected from nucleosides, nucleotides, and analogs and conjugates thereof In yet another embodiment, the drug comprises a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still another embodiment, the drug is selected from diuretics, vasodilators, inotropic agents, anti-arrhythmic agents, Ca⁺ channel blocking agents, anti-adrenergics/sympatholytics, and renin angiotensin system antagonists. In one embodiment, the drug is a VEGF inhibitor. VEGF antibody, VEGF antibody fragment, or another anti-angiogenic agent. Examples include an aptamer, such as MACUGEN™ (Pfizer/Eyetech) (pegaptanib sodium)) or LUCENTIS™ (Genetech/Novartis) (rhuFab VEGF, or ranibizumab), which could be used in the prevention of choroidal neovascularization (useful in the treatment of age-related macular degeneration or diabetic retinopathy). In yet a further embodiment, the drug is a prostaglandin, a prostacyclin, or another drug effective in the treatment of peripheral vascular disease. In still another embodiment, the drug is an angiogenic agent, such as VEGF. In a further embodiment, the drug is an anti-inflammatory such as dexamethasone. In one embodiment, a device includes both angiogenic agents and anti-inflammatory agents. A single device may include a single drug or a combination of two or more drugs.

The release of drug from a single reservoir may be tailored to provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e. pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release).

In one embodiment, the drug formulation within a reservoir comprises layers of a drug or drugs and a non-drug material, wherein the multiple layers provide pulsed drug release due to the intervening layers of non-drug. Such a strategy can be used to obtain complex release profiles.

In an alternative embodiment, the reservoirs may be used to store and control exposure of objects or materials other than drug formulations. For example, the reservoir contents may be a sensor or sensor component fixed inside each discrete reservoir. As used herein, a “sensing component” includes a component utilized in measuring or analyzing the presence, absence, or change in a chemical or ionic species, energy, or one or more physical properties (e.g., pH, pressure) at a site. Types of sensors include biosensors, chemical sensors, physical sensors, optical sensors, and pressure sensor. In one embodiment, the sensor could monitor the concentration of an analyte present in the blood, plasma, interstitial fluid, vitreous humor, or other bodily fluid of the patient. In one embodiment, a elongated device is provided having reservoir contents that include drug for release and a sensor/sensing component. For example, the sensor or sensing component can be located in a first reservoir and may operably communicate with a controller to control or modify the release characteristics of a drug from a second reservoir in the same or a separate implant device. See U.S. Pat. No. 6,551,838, which is incorporated herein by reference.

Discrete Reservoir Caps and Membrane Coverings

Reservoir openings may be closed off by at least one reservoir cap, membrane, film, or other structure. For example, each reservoir may further include a discrete reservoir cap. A reservoir cap covers the opening(s) of the reservoir to protect the reservoir contents (e.g., the drug formulation) until such time as release of the reservoir contents is desired. A reservoir cap is a thin film or other structure suitable for separating the contents of a reservoir from the environment outside of the reservoir. The reservoir caps are formed from a material or mixture of materials that degrade, dissolve, or otherwise disintegrate in vivo, or that do not degrade dissolve, or disintegrate, but become permeable in vivo to the drug molecules.

The device body or reservoir cap may include a wrap or coating of a semi-permeable or degradable material, such as a polymer, to control transport of molecules into or out of the reservoir when the device is in vivo. The reservoir caps for each of the reservoirs are designed to open at specified times, thereby delivering the reservoir content sequentially at pre-specified times. These reservoir caps may be independently disintegrated or permeabilized or groups of the reservoir caps can be actuated simultaneously. This reservoir opening may be passively controlled through the disintegrate of the reservoir cap. In a passive control system for example, the timing can be controlled by selecting the reservoir cap dimension, composition, and structure. Simultaneous actuation of the reservoir contents can be obtained by covering multiple reservoirs with reservoir caps of identical dimension, composition, and structure that will release the contents of different reservoirs at the same time.

The compositions of the reservoir caps may be selected from materials that will disintegrate in response to an environment existing in vivo in the patient or in response to a component contained in the reservoir. Examples of environmental conditions include, but are not limited to temperature, water, an electrolyte, and enzymes. Alternatively, the material of the reservoir cap may also be selected so that it will disintegrate when exposed to a form of energy that is applied to the patient from an external source or implanted internal source, such as acoustic (audible or ultrasonic) energy, magnetic energy, electromagnetic radiation (e.g., UV, visible, IR light, RF energy, or X-ray).

As used herein, the term “disintegrate” refers to degrading, dissolving, rupturing, fracturing or some other form of mechanical failure, as well as fracture and/or loss of structural integrity of the reservoir cap due to a chemical reaction or phase change (e.g., melting or transitioning from a solid to a gel), unless a specific one of these mechanisms is indicated. Hydrolytic decomposition is a preferred form of disintegration.

In preferred embodiments, the reservoir caps are selected to dissolve or biodegrade in vivo, without any intervention by the patient or caregiver. In one particular embodiment, the reservoir caps are formed of a biocompatible polymer, such as a poly(lactic acid), poly(glycolic acid), or poly(lactic-co-glycolic acid)s, as well as degradable poly(anhydride-co-imides), of a composition and thickness designed to disintegrate by hydrolysis in a prescribed timeframe, releasing the contents of the reservoir.

As used herein, the term “permeabilize” is used broadly to include without limitation some form of physical change that does not alter the chemical composition or dry mass of the membrane but changes the capability of the membrane to contain the reservoir contents. In a preferred embodiment of permeabilization, the polymer swells, changing its porosity without decomposition so that the reservoir contents are in contact with fluid external to the device and are releasable through the resulting open pore structure.

In a preferred embodiment, a discrete reservoir cap completely covers one of the reservoir's openings. In another embodiment, a discrete reservoir cap covers two or more, but less than all, of the reservoir's openings.

Representative examples of reservoir cap materials include polymeric materials and various types of semi-permeable membranes, and non-polymeric materials. In a preferred embodiment, the reservoir caps are non-porous and are formed of a bioerodible or biodegradable material, known in the art, such as a synthetic polymer, e.g., a polyester (such as PLGA), a poly(anhydride), or a polycaprolactone. The reservoir cap may be a multilayer structure. For example an inner layer may be porous or otherwise control diffusion once the outer, non-permeable layer has disintegrated. The reservoir caps of a single device may be made of different materials, may have different thicknesses, may have different degrees of cross-linking, or a combination thereof, for the purpose of opening different reservoirs at different times relative to one another.

Illustrative Embodiments of the Polymeric Reservoir Devices and Systems

The different embodiments of devices that can be created to use the implantable reservoir devices described herein can be understood with reference to the following non-limiting illustrations and descriptions of exemplary embodiments.

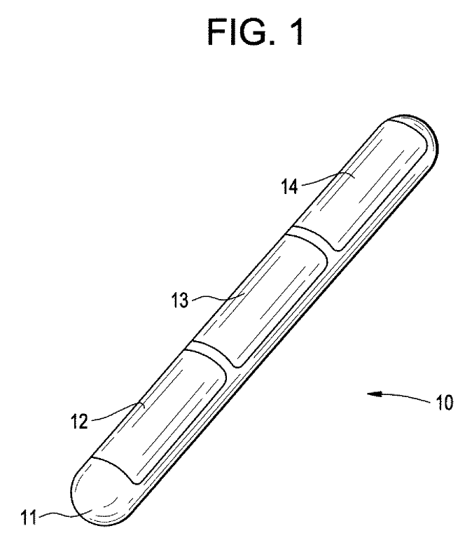

FIG. 1 is an external, perspective view of an exemplary embodiment of an assembled, implantable medical device. The device 10 of the exemplary embodiment includes an elongated body portion 11 which defines three discrete reservoirs and provides attachment points for respective reservoir caps 12, 13, and 14. The device 10 includes first and second rounded (e.g., hemispherical) end regions and a cylindrical intermediate region therebetween. In the illustrated embodiment, the reservoir caps are curved, with the convex side having a radius of curvature approximately equal to that of the intermediate region of the body portion. The number of reservoirs in a particular device may be varied depending upon the needs of the medical application. For example, for a vaccination application or gene therapy, the number of reservoirs may be three. For purposes of description herein, the illustrative example of three reservoirs and three reservoir caps will be used below to describe the implantable reservoir device, although other numbers of reservoirs may be dictated by the number of individual releases that are required for a particular drug release profile.

The reservoir caps 12, 13, and 14 may be formed of a biocompatible material that will disintegrate and/or permeabilize when exposed to particular conditions in vivo, and which may be tailored so that each disintegrates and/or permeabilizes at different rates or at the same rate.

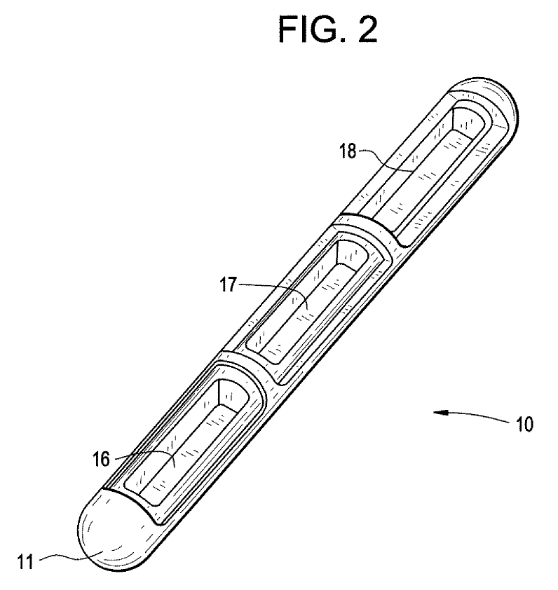

FIG. 2 shows the elongated body portion 11 without the other components of device 10, shown in FIG. 1. The elongated body portion 11 of this exemplary embodiment has three reservoirs 16, 17, and 18. The reservoirs 16, 17, and 18 may be filled with different drug formulations. Elongated body portion 11 includes contouring or other features that will mate with the peripheral edges of reservoir caps 12, 13, and 14 and allow secure attachment of the reservoir caps to the body portion 11. The reservoirs 16, 17, and 18 may have different shapes, may have different orientations (e.g., may have openings on opposing sides), and may vary (e.g., in size, shape, volume, etc.) from one another within a single device. In one case, the reservoirs may themselves be cylindrical in shape with a circular opening (e.g., made by laser drilling). Each reservoir 16, 17, and 18 may be integrally formed within the elongated body portion 11. A separate sealing layer is not required where, as here, the bottom surface of the reservoir is integrally formed with the sidewalls.

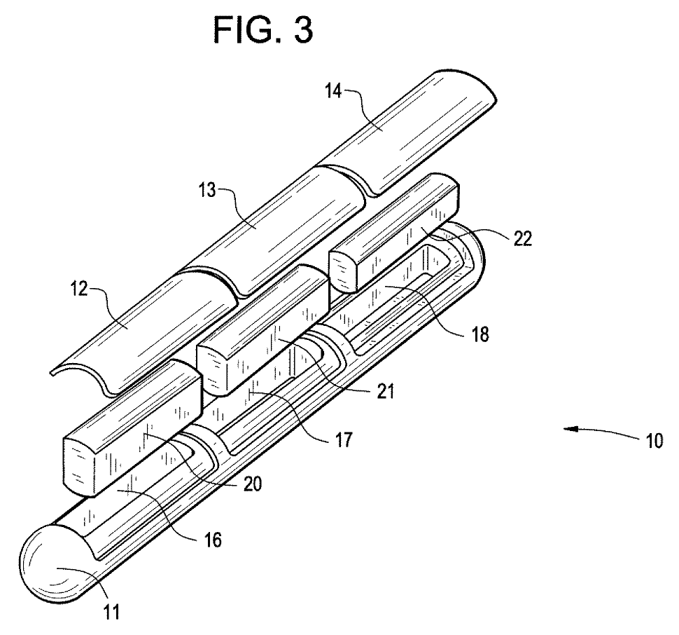

FIG. 3 is an exploded, perspective view of the device 10 that shows a elongated body portion 11 which defines the reservoirs 16, 17, and 18 and reservoir caps 12, 13, and 14. In one embodiment, the reservoirs 16, 17, and 18 each may have a volume of between about 1 μL and 10 μL (microliters). Drug formulations 20, 21, and 22 are disposed inside the reservoirs 16, 17, and 18, stored and protected from the environment external to the device until each of the respective reservoir caps 12, 13, and 14 disintegrates and/or become permeable as a consequence of exposure to particular conditions in vivo. The drug formulations 20, 21, and 22 may be identical or each may be formulated differently, e.g., a different drug, a different dose, or a different amounts or types of excipient materials. The release of these reservoir contents can be staggered by varying the composition, thickness, porosity, or degree of crosslinking of the different reservoir caps, by varying the formulation of the components themselves (such as by providing the active ingredient in formulation with different matrix materials or within different layers or gradients within each reservoir) or a combination thereof. The reservoir caps on a device may be different or the same or in any combination to control the desired delivery of the reservoir contents. It should also be noted that the reservoir is a sealed enclosure between the body portion and the reservoir caps despite any appearance to the contrary suggested by the “cut-away” cross-section view of FIGS. 2 and 3.

In a particular preferred embodiment, the reservoir caps may be positioned in multiple locations around reservoirs that are contained within a substantially rod-shaped device formed of a poly lactide co-glycolide polymer of a composition and thickness designed to disintegrate by hydrolysis in a prescribed timeframe, releasing the drug formulation contents of the reservoir. The configuration is similar to that depicted in FIG. 2, but the reservoir includes multiple, substantially opposed openings in the device body. In this way, the top and bottom (and/or sides) of the elongated device portion) may have openings that are closed off by discrete reservoir caps or a membrane wrap. The multiple discrete reservoir caps associated with a particular reservoir may disintegrate in vivo substantially simultaneously, thereby effectively increase the surface area available for diffusion of drug from the reservoir, and advantageously providing local release of the drug to tissues in multiple or substantially all directions from the elongated device body. This may improve the rate of release of drug over that provided from a single-opening-on-a-single-side reservoir.

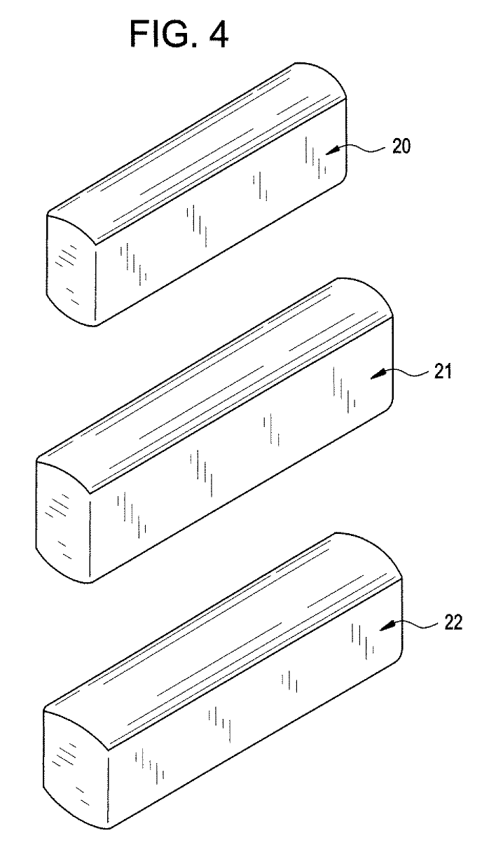

FIG. 4 illustrates exemplary embodiments of pre-formed drug formulations 20, 21, and 22 of device 10. Each may be formulated to be substantially identical to the others, or, alternatively, some or all of the drug formulations may be different from others. The shape and composition of each pre-form may be varied depending, for example, on the amount (e.g., volume) of drug to be delivered from each reservoir. A single pre-form may represent a single dose or a partial dose of a drug, depending on the particular dosing schedule desired and whether multiple reservoirs are to be opened simultaneously or in a temporally overlapping fashion. In one embodiment, drug formulation 20 has a volume of about 7.8 μL, drug formulation 21 has a volume of about 7.3 μL, and drug formulation 22 has a volume of about 6.4 μL.

In in vivo operation, the reservoir caps 12, 13, and 14 become permeabilized or disintegrate to unblock/uncover the reservoir openings and expose the reservoir contents (e.g., the drug formulation) to fluids and tissue located adjacent the implanted medical device, allowing the drug to diffuse from the reservoir and reach local tissues for treatment or prophylaxis. In one embodiment, bodily fluids enter the opened reservoirs and cause a solid drug formulation to dissolve into solution, thereby facilitating drug delivery.

In a preferred embodiment, the elongated body portion 11 includes a biocompatible metal, such as titanium, that is radioopaque, allowing more accurate positioning of the device within target tumors, organ, or other sites in the body which require precise local administration of a therapeutic agent. The elongated body portion may be fabricated substantially entirely of a metal, or the elongated body portion may include a biocompatible metal and a polymeric material. For example, the elongated body portion of the device may have a polymeric core and a metal coating, so that the edges of the body portion can be readily discerned in vivo by x-ray. In another example, the device may have both a metal portion and a polymeric portion.

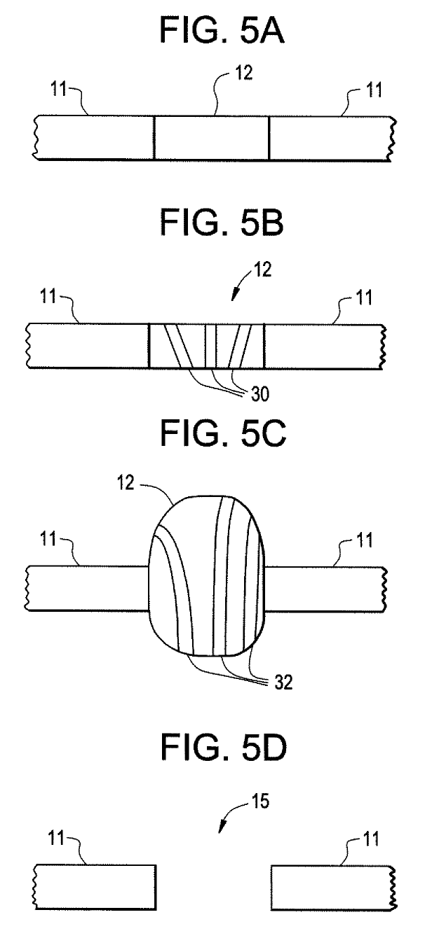

The reservoir caps 12, 13, and 14 may disintegrate by any of several different mechanisms to permit release of the reservoir contents 20, 21, and 22 from the reservoirs. FIGS. 5a-5d are cross-sectional views that illustrate non-limiting examples of a reservoir cap undergoing disintegration. FIG. 5a illustrates reservoir cap 12 in its initial, undisintegrated state. FIG. 5b shows reservoir cap 12, which has begun to degrade, creating channels 30 in the structure capable of permitting ingress of bodily fluids and diffusion of drug from the reservoir. FIG. 5c shows reservoir cap 12, wherein the polymeric reservoir cap has absorbed fluid in vivo causing the reservoir cap to swell and create pores/channels 32 therein. FIG. 5d illustrates the reservoir opening 15 remaining in elongated body portion 11 after complete disintegration or dissolution of reservoir cap 12. A reservoir may undergo combinations of these illustrated states over the course of implantation. It is understood that the release of drug is not limited to the exemplary embodiments disclosed in FIGS. 5a-5d.

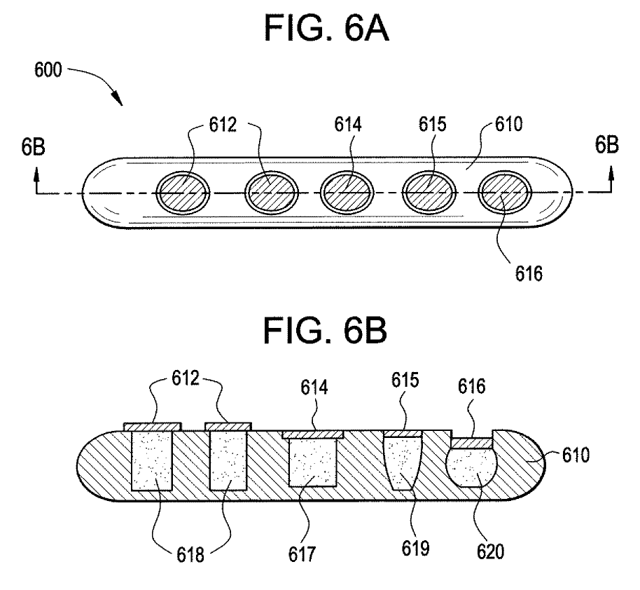

As illustrated in FIGS. 6a-b, in top-view and cross-sectional view, respectively, elongated implant device 600 includes a body portion 610 having a linear array of five reservoirs. FIG. 6b shows some of the possible variations in reservoir cap position relative to the exterior surface of the body portion and reservoir opening, as well as some of the possible shapes of the reservoirs. Reservoirs 618 are covered by reservoir caps 612, which extend from the exterior surface of the longitudinal side of device body 610. Reservoirs 617 and 619 are covered by reservoir caps 614 and 615, respectively, the tops of which are flush with the exterior surface of the longitudinal side of device body 610. Reservoirs 617 and 618 have straight interior side walls, and reservoir 619 has side walls that taper toward the bottom of the reservoir. Reservoir 620 has rounded side walls and is covered by reservoir cap 616, which is recessed below the exterior surface of the longitudinal side of device body 610. The particular arrangement and combination of reservoir and reservoir cap may be selected based on drug release kinetics and dosing needs, manufacturing considerations, and device implantation considerations.

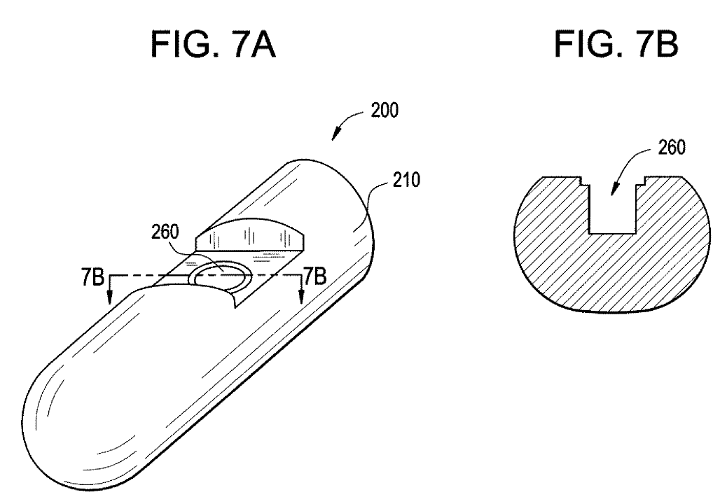

FIG. 7a illustrates a perspective view of an embodiment of the device 200 wherein the reservoir 260 is recessed into the elongated body portion 210. FIG. 7h shows a cross-section of the body and reservoir. Recession of the reservoir 260 may provide flexibility in the design of the reservoir cap for material design and dimension to control the degradation. Recession of the reservoir cap may permit the reservoir cap to make a smooth interface with the body at the reservoir opening, e.g., the joint/interface of the two components can be planar even though the outer surfaces are curved.

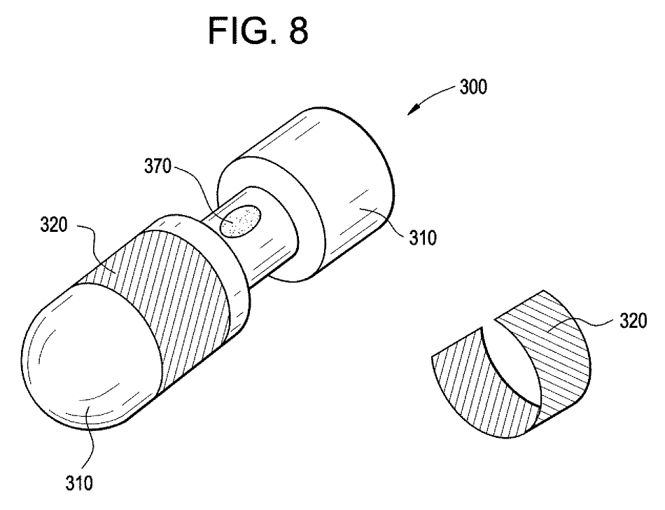

In another embodiment illustrated in FIG. 8, in a perspective and partially exploded view, an elongated implant device 300 which includes two reservoirs 370 (only one is visible) located in elongated body 310. Reservoir 370 may have openings in more than one direction about the longitudinal axis of the elongated body 310. For example, the reservoir 310 may extend from the top to the bottom of the recessed portion of the substrate 310 to allow drug to be delivered in a plurality of lateral directions. A single reservoir cap may be shaped and dimensioned to cover more than one of the reservoir openings. In the illustrative embodiment, the reservoir caps 320 extend about the circumference of the elongated body 310 in a groove in the body and provide a flush fitting with the longitudinal exterior surface to cover the openings in reservoirs 370. The reservoir cap may be secured to the substrate in essentially any manner known in the art for securing structures together, e.g., using a biocompatible adhesive, fasteners, and/or relying on frictional engagement between surfaces of the components.

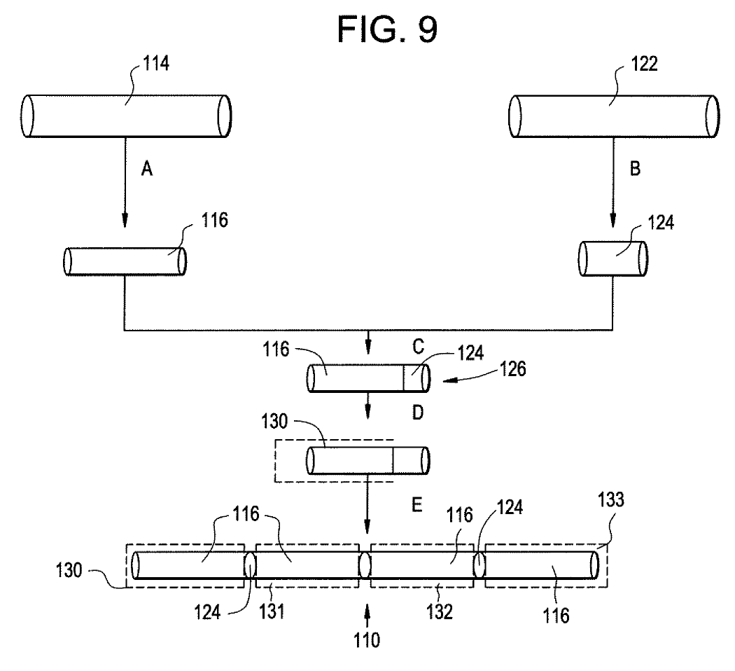

FIG. 9 illustrates a process for making one embodiment of an elongated implantable medical device 110. The assembled device 110 includes a plurality of solid units of a drug formulation, seal members, and controlled-release membrane coatings. The process may include the steps of casting, extruding, or otherwise forming a source rod of a drug formulation 114, which provides a source of solid units 116 of a drug formulation. Step A is dicing or slicing the source rod into multiple solid units. The process also may include casting, extruding, or otherwise forming a source rod of a sealing material 122, which provides a source of sealing members 124. Step B is dicing or slicing the source rod into multiple sealing members. At step C, a solid unit of a drug formulation 116 is connected to a sealing member 124, to form a formulation/seal component 126. The connection may be made using a biocompatible adhesive, fastener, or another technique known in the art. At step D, the formulation formulation/seal component 126, or at least the solid unit 116 thereof, is coated with a membrane material 130. The membrane's composition and thickness are selected to disintegrate or permeabilize in vivo at the appropriate time and rate to provide a predetermined release profile for the drug.

Step D may be repeated on other solid units 116 of drug formulation with different membrane coatings 131, 132, and 133. As shown in Step E, a plurality of coated formulation/seal components may be assembled together to form the elongated implantable device 110. The device 110 therefore includes multiple, discrete doses of drug formulation 116, with each dose being independently, passively controlled to release the drug in vivo at a predetermined schedule and rate, controlled by an membrane coating 130, 131, 132, and 133 associated therewith.

The sealing member serves to shield an end portion of a second unit on one side of the member even after the first unit on the other side of the member has had its membrane coating disintegrated or permeabilized, and whether or not the first unit has disintegrated or release all of its drug contents.

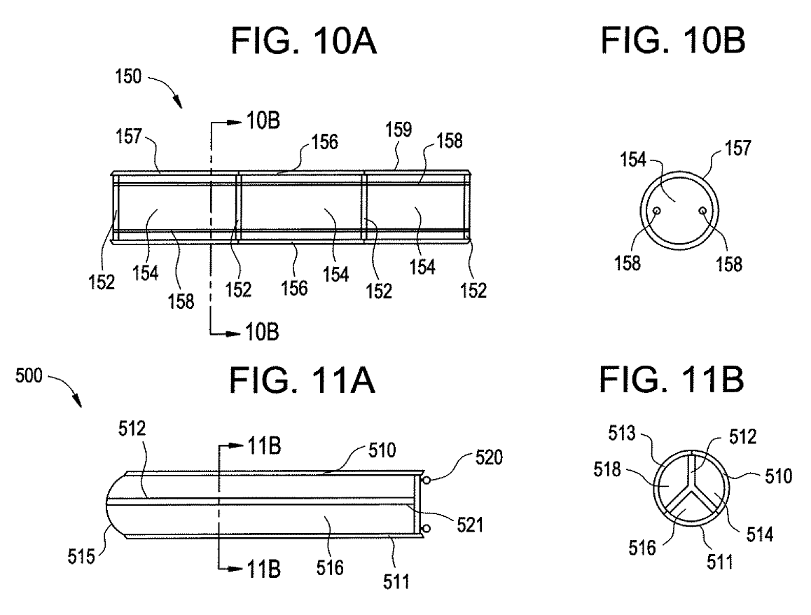

FIGS. 10a-b illustrate, in a perspective/transparent view and in a cross-sectional view, another non-limiting example of an elongated implant device 150. The device 150 includes a plurality of impermeable discs 152 connected with two support rods 158. The rods may be positioned about the circumference of the impermeable discs 152. One or any number of support rods may be used to support the discs. The volume between the impermeable discs 152 defines discrete reservoirs. Each reservoir is filled with drug formulation 154, and covered by a different membrane material 156, 157, and 159. The membrane material initially serves as the longitudinal walls of the elongated device. Release of the drug from the drug formulation is controlled by disintegration or permeabilization of the membrane material. Release of drug can occur in all directions to the elongated device, as the reservoir “opening” circumscribes the device body.

FIGS. 11a-b illustrate, in cross-sectional views, an elongated medical implant 500 which includes three elongated, longitudinally oriented reservoirs respectively containing drug formulations 514, 516, and 518, which are separated from one another by a centrally disposed three-member barrier and support structure 512. The reservoirs 514, 516, and 518 are bounded at their ends by a nose cap 515 and an end cap 521, which includes a suture loop 520. The reservoirs 514, 516, and 518 are bounded at the longitudinal sides of the device by membrane materials 510, 511, and 513. Release of the drug from the drug formulation is controlled by disintegration or permeabilization of the membrane materials. The nose cap and the end cap preferably are attached to the barrier and support structure 512. The nose cap, end cap, and support structure may be non-degradable, or degradable if their degradation is slow enough to not significantly impact the preselected, desired drug release characteristics of the device. These parts may be made of a metal, ceramic, or polymer material that is substantially impermeable. The longitudinal reservoirs may or may not extend the full length of the device. It also is envisioned that the implant device could be varied to have two, four, or other numbers of reservoirs, other shapes of nose and end caps, and that features of the various embodiments illustrated herein may be interchanged for different applications.

Making the Devices

The basic methods of fabricating and assembling the elongated body portion described herein are known or can be readily adapted from techniques known in the art. Reservoirs may be created in the device body simultaneously with formation of the device body, or they may be formed in the device body after the device body is made.

Representative fabrication techniques include MEMS fabrication processes, microfabrication processes, or other micromachining processes, various drilling techniques (e.g., laser, mechanical, and ultrasonic drilling), electrical discharge machining (EDM), and build-up or lamination techniques, such as LTCC (low temperature co-fired ceramics) Microfabrication methods include lithography and etching, injection molding and hot embossing, electroforming/electroplating, microdrilling (e.g., laser drilling), micromilling, electrical discharge machining (EDM), photopolymerization, surface micromachining, high-aspect ratio methods (e.g., LISA), micro stereo lithography, silicon micromachining, rapid prototyping, and DEEMS (Dry Etching, Electroplating, Molding). Reservoirs may be fabricated into metal body portions by techniques known in the art, including laser etching, laser jet etching, micro-EDM, oxide film laser lithography, and computerized numerical control machining.

The surface of the reservoir optionally may be treated or coated to alter one or more properties of the surface. Examples of such properties include hydrophilicity/ hydrophobicity, wetting properties (surface energies, contact angles, etc.), surface roughness, electrical charge, release characteristics, and the like.

U.S. Pat. No. 6,808,522; U.S. Pat. No. 6,123,861; U.S. Pat. No. 6,527,762; and U.S. Pat. No. 6,976,982, which are hereby incorporated by reference, describe micromolding and other techniques for making certain reservoir device bodies and caps. These methods may be modified and adapted, based on the teachings herein, to make the elongated implant devices described herein.

Implanting/Using the Devices

The devices described herein can be used in a wide variety of applications. Preferred applications include the administration of one or more drugs to a patient in need thereof. In a preferred embodiment, the device is an implantable medical device. The implantable medical device can take a wide variety of forms and be used in a variety of therapeutic, prophylactic, or diagnostic medical applications. In a preferred embodiment, the devices store and release an effective amount of at least one drug formulation over an extended period, e.g., between 1 and 12 months.

The device may be implanted into a patient (such as a human or other vertebrate animal) using standard surgical or more preferably a minimally invasive implantation technique, such as injection through a needle, trocar, cannula, catheter, or the like. Ultrasound, nuclear magnetic resonance, virtual anatomic positioning systems, or other imaging techniques may be employed to confirm proper positioning of the implant. In various embodiments, the administration system may include a catheter, wire, tube, endoscope, or other mechanism capable of reaching the desired recipient anatomic site through an incision, puncture, trocar, or through an anatomic passageway such as a vessel, orifice, or organ lumen, or trans-abdominally or trans-thoracically. In various embodiments according to the present invention, the delivery system may be steerable by the operator.

In one embodiment, the implant device may be dimensioned for delivery from a 6 to 26 gauge (approximately 0.439 mm to 0.241 mm nominal ID) needle. Larger or smaller gauges of modified needles may also be used to accommodate various sized drug delivery implant devices described herein. It may be preferable to implant 5 devices that have larger diameters using means known in the art other than needles. In certain embodiments, the drug delivery implant described herein may be delivered using apparatus and techniques described, for example, in U.S. Pat. No. 7,214,206 and U.S. Pat. No. 7,104,945, which are incorporated herein by reference.

Drug may then be passively released locally at the tissue site of implantation at 10 a preselected delivery profile (e.g., dosing schedule) based on the design of the particular device as prescribed by the patient's physician.

Modifications and variations of the methods and devices described herein will be obvious to those skilled in the art from the foregoing detailed description. Such modifications and variations are intended to come within the scope of the appended claims.

We claim:

1. An implantable medical device for controlled drug delivery comprising:
an elongated body dimensioned to facilitate minimally invasive and complete implantation into a patient, wherein the elongated body portion comprises a first, closed end portion, a second, closed end portion and an intermediate portion disposed between and connected to the first closed end portion and the second closed end portion;
two or more discrete reservoirs disposed in the elongated body portion, each reservoir having one or more openings;
a drug formulation, which comprises at least one drug, disposed in the two more discrete reservoirs; and
reservoir caps closing off the one or more openings of each reservoir,
wherein release of the drug in vivo is controlled at least in part by the in vivo disintegration or permeabilization of the discrete reservoir caps.

2. The device of claim 1, wherein the intermediate portion is substantially tubular in shape, and the first closed end portion comprises a rounded end.

3. The device of claim 2, wherein the device is suitable for passing through a trochar, hollow needle, or catheter.

4. The device of claim 1, wherein the elongated body is formed of a polymer.

5. The device of claim 1, wherein the elongated body is formed of a metal.

6. The device of claim 1, wherein the reservoir caps comprises a polymer which disintegrates in vivo.

7. The device of claim 1, wherein the drug formulation is in a solid form.

8. The device of claim 7, wherein the drug formulation comprises microparticles or nanoparticles of a drug.

9. The device of claim 7, wherein the drug formulation in each reservoir is in a monolithic structure form.

10. The device of claim 1, wherein the drug comprises a protein, a peptide, or a nucleic acid.

11. The device of claim 1, wherein the discrete reservoirs are microreservoirs.

12. The device of claim 1, wherein the drug is a vaccine.

13. The device of claim 1, wherein the drug is a chemotherapeutic agent or a hormone.

14. The device of claim 1, wherein the device comprises a linear array of three or more reservoirs, each containing a drug formulation and having a discrete reservoir cap closing off at least one opening of each reservoir.

15. The device of claim 1, wherein at least two of the reservoir caps differ from one another in composition or thickness or both.

16. The device of claim 1, wherein at least one of the reservoirs extends into the elongated body in a substantially traverse direction to a depth which is more than 50% of the maximum traverse width of the intermediate body portion.

17. The device of claim 1, wherein at least one of the reservoirs has a first opening and an opposing second opening located distal to one another in the traverse direction in the intermediate portion of the elongated device body.

18. The device of claim 1, wherein the elongated body defines a plurality of reservoirs about the circumference of the device and wherein the reservoirs extend the substantial length of the elongated body.

19. An implantable medical device for controlled drug delivery comprising,
a first solid unit of a drug formulation which comprises at least one drug, the first solid unit have a proximal end, a distal end, and one or more longitudinal exterior surfaces therebetween;
a second solid unit of the drug formulation which comprises the at least one drug, the second solid unit have a proximal end, a distal end, and one or more longitudinal exterior surfaces therebetween;
a layer of a first membrane material coating the longitudinal exterior surfaces of the first solid unit;
a layer of a second membrane material coating the longitudinal exterior surfaces of the second solid unit; and
a substantially impermeable seal member sandwiched between the proximal end of the first solid unit and the distal end of the second solid unit, such that the first solid unit, seal member, and second solid unit are connected together aligned in an elongated rod shaped form, which is dimensioned to facilitate minimally invasive and complete implantation into a patient,
wherein release of the drug in vivo is controlled at least in part by the in viva disintegration or permeabilization of the first and second membrane materials.

20. The implantable medical device of claim 19, wherein release of the drug from the first solid unit occurs at a different time or rate than release of the drug from the second solid unit.

21. The implantable medical device of claim 19, wherein the first and second units each have a cylindrically shaped exterior surface, and the seal member is disk shaped.

22. The implantable medical device of claim 19, wherein the seal member comprises a non-degradable polymer.

23. The implantable medical device of claim 19, wherein the first membrane material, the second membrane material, or both membrane materials comprise a biodegradable polymer.

24. The implantable medical device of claim 19, wherein drug formulation further comprises one or more pharmaceutically acceptable excipient materials.

25. The implantable medical device of claim 19, which comprises three or more of the membrane coated solid units of drug formulation and two or more of the seal members.

26. The implantable medical device of claim 19, further comprising a plurality of rods connecting the seal members.

27. A method of the administration of a drug to a patient comprising:
injecting the implantable medical device of claim 1 into a tissue site in a patient; and
releasing the drug from the medical device to the tissue site.

28. The method of claim 27, wherein the step of injecting is subcutaneous or intramuscular.

29. The method of claim 27, wherein the tissue site is a tumor mass.

30. The method of claim 27, wherein the tissue site is in the brain, the peritoneal cavity, an intracranial cavity, or the pleural space.

31. The method of claim 27, wherein the step of injecting comprises passing the implantable medical device through the hollow bore of a needle, cannula, catheter, or trocar.
